(19) United States
(12) Patent Application Publication
(10) Pub. No.: US 2023/0346582 A1
(43) Pub. Date: Nov. 2, 2023

(54) STENT-DEPLOYMENT ASSEMBLIES WITH LOCKING MECHANISMS AND METHODS OF ASSEMBLY

(71) Applicant: ENDO GI MEDICAL LTD., Nazareth (IL)

(72) Inventors: Omri NAVEH, Ramat Yshay (IL); Elad EINAV, Salit (IL); Ronny BARAK, Tel Aviv (IL)

(21) Appl. No.: 18/025,623

(22) PCT Filed: Sep. 14, 2021
(86) PCT No.: PCT/IB2021/058386

§ 371 Date: Mar. 9, 2023

Related U.S. Application Data
(63) Continuation-in-part of application No. 17/140,001, filed on Jan. 1, 2021, now Pat. No. 11,744,694.
(60) Provisional application No. 63/078,539, filed on Sep. 15, 2020.
(60) Provisional application No. 63/078,539, filed on Sep. 15, 2020.
(60) Provisional application No. 62/956,294, filed on Jan. 1, 2020.

(51) Int. Cl. A61F 2/97

(52) U.S. Cl. CPC A61F 2/97; A61F 2002/9511

(57) ABSTRACT

A stent-deployment assembly includes an elongated stent-conveyance tube comprising a hollow longitudinal lumen configured to have a guidewire traverse longitudinally therethrough. The stent is arranged to surround a first stent-conveyance tube segment and a pushing tube surrounds a second stent-conveyance tube segment that is proximally displaced from the first stent-conveyance tube segment. A first locking member is engaged with the elongated stent-conveyance tube, and a second locking member includes a first portion arranged to transversely traverse the pushing tube and a second portion constrained within an interior volume of the stent by the presence of the first locking member.

### CROSS-REFERENCE TO RELATED APPLICATIONS

This patent application claims the benefit of U.S. Provisional Patent Application No. 63/046,746 filed on Sep. 15, 2021, which is incorporated herein by reference in its entirety. This patent application is a continuation of U.S. patent application Ser. No. 17/140,001 filed on Jan. 1, 2021, which is incorporated herein by reference in its entirety. U.S. patent application Ser. No. 17/140,001 claims the benefit of U.S. Provisional Patent Application No. 62/956,294 filed on Jan. 1, 2021, which is incorporated herein by reference in its entirety.

### FIELD OF THE INVENTION

Embodiments of the present invention relate generally to medical devices and more particularly to methods and apparatus for deploying stents in a lumen of a subject.

### BACKGROUND

Stents are typically deployed within a lumen of a body of a subject for various reasons. In some cases, a stent is deployed within a lumen in order to widen a narrowed section of the lumen. In one example, insertion of a stent into a bile duct is used to treat obstructions and strictures that occur in the bile duct. A stent is typically a tube-like structure that can be used to support a narrowed part of a lumen and inhibit the reformation of the stricture. A tube or catheter is often used to deploy stents, and a guidewire is often used to aid in guiding the stent to its targeted deployment location within the lumen. Locking mechanisms are used to control movement of the stent relative to the tube or catheter during deployment, but available devices require additional steps to release or disengage the locking mechanisms.

### SUMMARY OF THE INVENTION

According to embodiments disclosed herein, a stent-deployment assembly for use with a guidewire comprises: (a) an elongated stent-conveyance tube comprising a guidewire-retaining segment, the guidewire-retaining segment (i) including a lengthways laterally-breachable portion and (ii) being configured for having the guidewire traverse longitudinally therethrough; (b) a stent arranged to surround a stent-conveyance tube segment that is proximally displaced from the guidewire-retaining segment; (c) a pushing tube surrounding the stent-conveyance tube segment and proximally displaced from the stent; and (d) a proximally-withdrawable locking mechanism, proximally engaging the stent with the pushing tube so as to constrain distal movement of the stent relative to the pushing tube, wherein, when movement of the stent is externally constrained, a proximal-direction withdrawal of the stent-conveyance tube is effective to (i) cause the guidewire to breach the laterally-breachable portion of the guidewire-retaining segment so as to decouple the guidewire from the tube without manipulation of the guidewire, and (ii) disengage the proximally-withdrawable locking mechanism.

In some embodiments, it can be that a proximal-direction withdrawal of the stent-conveyance tube effective to cause the guidewire to breach the laterally-breachable portion of the guidewire-retaining segment can be effected by applying a proximal-withdrawal force of at least 100 grams and no more than 20 kg.

In some embodiments, the stent-deployment assembly can additionally comprise the guidewire.

In some embodiments, the proximally-withdrawable locking mechanism can include a first locking member proximally engaged with the elongated stent-conveyance tube. In some embodiments, the first locking member can be integrally formed with the elongated stent-conveyance tube. In some embodiments, the first locking member can be fixedly attached to the elongated stent-conveyance tube. In some embodiments, the first locking member can be detachably attached to the elongated stent-conveyance tube.

In some embodiments, the proximally-withdrawable locking mechanism can include a second locking member comprising a loop portion. In some embodiments, the second locking member can transversely traverse the pushing tube. In some embodiments, a portion of the second locking member can be constrained within an interior volume of the stent by the presence of the first locking member.

In some embodiments, the disengaging of the proximally-withdrawable locking mechanism can include proximally withdrawing the first locking member so as to no longer constrain the portion of the second locking member.

A method is disclosed, according to embodiments, for assembling a stent-deployment apparatus. The method comprises: (a) providing a stent-conveyance tube comprising a distal guidewire-retaining segment, the stent-conveyance tube having a first locking member of a proximally-withdrawable locking mechanism engaged with the stent-conveyance tube; (b) arranging a pushing tube around a first stent-conveyance tube segment; (c) arranging a stent around a second stent-conveyance tube segment that is proximally displaced from the guidewire-retaining segment and distally displaced from the first stent-conveyance tube segment; and (d) installing a second locking member, wherein the installing includes (i) engaging the second locking member with the pushing tube and (ii) constraining a loop portion of the second locking member within an interior volume of the stent to be constrained therein by the presence of the first locking mechanism.

In some embodiments, the method can additionally comprise: passing the guidewire longitudinally through the distal guidewire-retaining segment of the stent-conveyance tube.

In some embodiments, it can be that the passing through of the guidewire is not factory-performed and all other steps are factory-performed.

In some embodiments, the first locking member can be integrally formed with the stent-conveyance tube. In some embodiments, the first locking member can be fixedly attached to the stent-conveyance tube. In some embodiments, the first locking member cam be detachably attached to the stent-conveyance tube.

According to embodiments of the invention, a stent-deployment assembly comprises: (a) an elongated stent-conveyance tube comprising a hollow longitudinal lumen configured to have a guidewire traverse longitudinally therethrough; (b) a stent arranged to surround a first stent-conveyance tube segment; (c) a pushing tube surrounding a second stent-conveyance tube segment that is proximally displaced from the first stent-conveyance tube segment; (d) a first locking member engaged with the elongated stent-conveyance tube; and (e) a second locking member comprising a first portion arranged to transversely traverse the pushing tube and a second portion constrained within an interior volume of the stent by the presence of the first locking member.

In some embodiments, the first and second locking members can be effective in combination to constrain distal movement of the stent relative to the pushing tube.

In some embodiments, when movement of the stent is externally constrained, a proximal-direction withdrawal of the stent-conveyance tube can be effective to cause the second locking member to no longer be constrained by the first locking member.

In some embodiments, the first locking member can be integrally formed with the stent-conveyance tube. In some embodiments, the first locking member can be fixedly attached to the stent-conveyance tube. In some embodiments, the first locking member cam be detachably attached to the stent-conveyance tube.

### BRIEF DESCRIPTION OF THE DRAWINGS

The invention will now be described further, by way of example, with reference to the accompanying drawings, in which the dimensions of components and features shown in the figures are chosen for convenience and clarity of presentation and not necessarily to scale. In the drawings:

FIG. 1 is a schematic illustration of a distal portion of a stent-conveyance tube, shown engaged with a guidewire, in accordance with embodiments of the present invention.

FIGS. 2A and 2B are schematic illustrations of a stent assembly and its deployment in a lumen of a human subject, in accordance with embodiments of the present invention.

FIG. 2C is a perspective view of a distal portion of a stent-conveyance tube in accordance to embodiments of the present invention.

FIG. 3 shows a flowchart of a method for deploying a stent in a lumen of a human subject, in accordance with embodiments of the present invention.

FIGS. 4A, 4B and 4C are schematic illustrations depicting a general overview of a method for deploying a stent in a lumen of a subject, in accordance with embodiments of the present invention.

FIG. 4C is a schematic illustration of a distal portion of a stent-conveyance tube, shown engaged with a guidewire, during proximal withdrawal of a stent-conveyance tube, in accordance with embodiments of the present invention.

FIGS. 5A, 5B and 5C are schematic illustrations of a proximally-withdrawable locking mechanism engaging a stent with a pushing tube, in accordance with embodiments of the present invention.

FIG. 6 is a schematic perspective view of a stent-deployment assembly, in accordance with embodiments of the present invention.

FIGS. 7A and 7B show flowcharts of method steps for assembling a stent-deployment apparatus, in accordance with embodiments of the present invention

### DETAILED DESCRIPTION OF EMBODIMENTS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice. Throughout the drawings, like-referenced characters are generally used to designate like elements.

According to embodiments, a stent assembly includes a stent mounted on a catheter tube adapted, e.g., for conveying the stent to a target deployment location within a lumen of a human subject.

The scope of the present invention includes stent assemblies intended for use in any suitable lumen in the human body. For example, techniques and apparatus described herein may be used in connection with stents for use in a urethra, and/or in a ureter, and/or in a pancreatic duct, and/or in an esophagus, and/or in a trachea of a subject. Additionally, or alternatively, techniques and apparatus described herein may be used in connection with prostatic stents. Additionally, or alternatively, techniques and apparatus described herein may be used in connection with biliary stents, i.e., stents used to to maintain flow viability of a bile duct.

The assembly is configured for advancement along a guidewire which is typically inserted into the subject's body in advance of deploying the stent. A distal end of the guidewire is disposed within the target lumen, and the proximal end remains outside the body. In the case of employing short-wire systems, the guidewire can be externally locked. The terms ‘distal’ and ‘proximal’ are used throughout this disclosure and the appended claims as follows: ‘distal’ means further into the body (along an insertion path) from a point of entry into the body, while ‘proximal’ means closer to the point of entry into the body. Where the terms are used in reference to apparatus outside of a patient's body, a distal portion or distal end is that portion or end of the apparatus configured to be inserted into the body first, while a proximal end or proximal portion is either inserted last or may never be inserted (as in the case of a guidewire, for example). Additionally, when used relatively, e.g. ‘distally displaced from’ or ‘proximal of’, the meaning is, respectively, closer to the distal end than’ or ‘closer to the proximal end than’.

As will be further described hereinbelow and in the accompanying figures, a catheter tube (which can be alternatively called, equivalently, ‘guide tube,’ ‘stent-conveyance tube,’ or, simply, ‘tube’) for conveyance of the stent is disclosed as having, at or near a distal tip, arrangements for engaging a guidewire. Apertures are provided on either end of a longitudinal guidewire-engaging or guidewire-retaining segment of the tube, and the guidewire can be threaded through these apertures so as to traverse the interior of the guidewire-retaining segment of the tube. The guidewire does not interiorly traverse the tube segment proximal to the guidewire-retaining segment, and thus ‘departs’ the interior of the tube, at least temporarily, at the proximal aperture. The distal aperture can be at the distal tip of the tube, or it can be displaced proximally from the tip. It is preferable that the distal aperture faces distally, i.e., faces in a direction in which the distal tip is facing, within 15° of that direction, or within 30° of that direction, or within 45° of that direction. The proximal aperture preferably faces proximally, i.e., faces in a direction opposite to the direction in which the distal tip is facing, or within 15° of that ‘opposite’ direction, or within 30° or within 45° of that ‘opposite’ direction. Thus, when the guidewire exits the proximal aperture, it is directed to continue alongside the tube (and alongside the stent that surrounds the tube) proximal to the proximal aperture.

With the guidewire passing through the interior of the guidewire-retaining segment, the tube can be advanced along the length of the guidewire with little resistance from the guidewire, for example pushed forward by an additional stent engaged with the guidewire or by a ‘pusher’ catheter (also called a ‘pushing tube’) engaged with the guidewire. The stent can be mounted on the tube before or after the tube is engaged with the guidewire, so as to surround a segment of the tube that is proximal to the guidewire-retaining segment. There can be a gap between the guidewire-retaining segment and the stent-carrying segment.

The configuration in which the stent is mounted on the tube so as to surround a segment that is proximal to the guidewire-retaining segment, and in which the tube is engaged with the guidewire in that the guidewire passes through the interior of the guidewire-retaining segment, is referred to herein as the ‘stent-advancement configuration’ of the stent assembly.

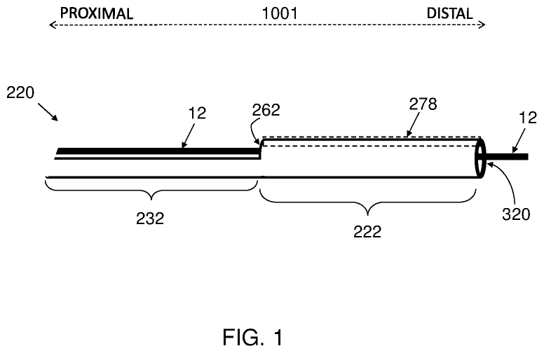

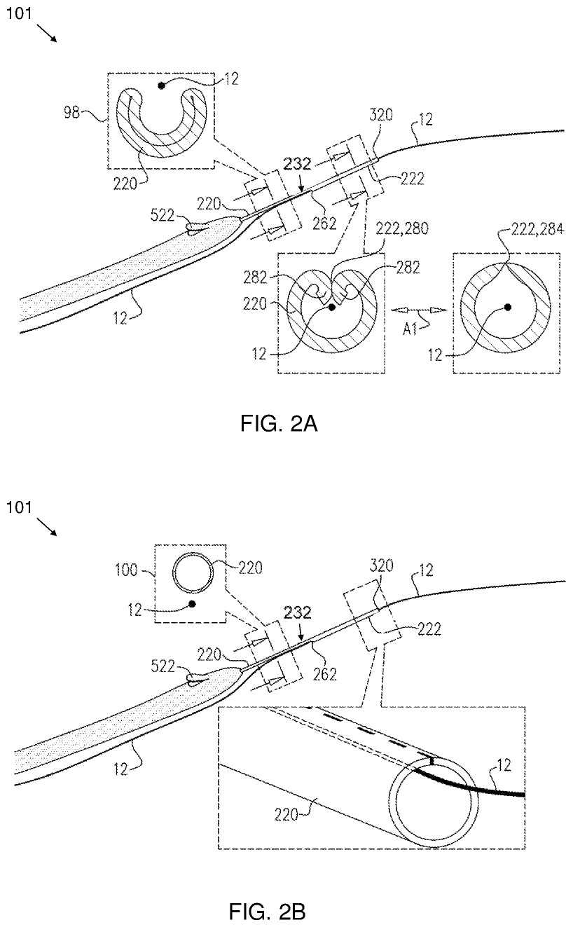

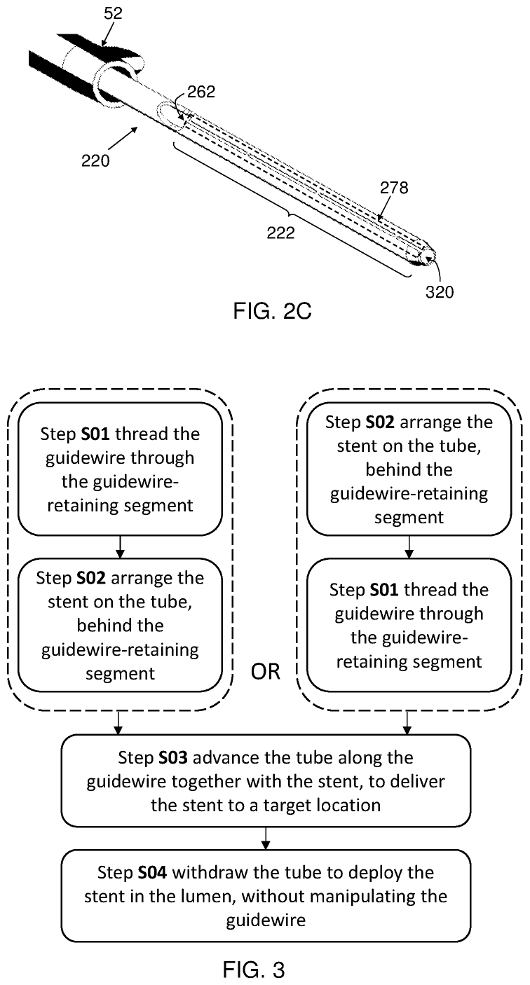

The guidewire-retaining segment is configured to retain the guidewire therewithin during the advancement of the stent into the body lumen in the stent-advancement configuration. In some embodiments, the relative longitudinal stability of the position of the stent relative to the tube is accomplished using a locking stent as will be discussed hereinbelow. The guidewire-retaining segment has a lengthways, laterally breachable portion, making the guidewire-retaining segment laterally breachable by the guidewire. The guidewire-retaining segment of the tube is designed to be laterally breached by the guidewire when a shearing force is applied, beginning at the proximal aperture when the tube is proximally withdrawn once the stent is deployed and anchored in the lumen (and any locking system is ‘unlocked’). The laterally-breachable portion can be a weakened or pre-breached sidewall of the guidewire-retaining portion, as will be discussed in greater detail hereinbelow with respect to FIGS. 1 and 2A-2B.

Once the stent has been advanced to a target stent-deployment location in the lumen of the patient's body, e.g., the bile duct, the stent-conveyance tube can be withdrawn proximally so as to leave the stent deployed in the lumen. The stent is preferably self-anchoring with one or more anchor flaps maintaining the position of the stent against the force used to withdraw the tube, such that the stent slides off the distal end of the tube when the tube is withdrawn. Once the proximal aperture of the guidewire-retaining reaches the edge of the stent, the guidewire exiting the proximal aperture is trapped against the proximal aperture by the unmoving stent, and the resulting shearing force causes the guidewire to breach the laterally-breachable portion of the guidewire-retaining segment. Continued application of the force causes the guidewire to laterally exit the breached guidewire-retaining segment and thus be disengaged or decoupled therefrom. The force necessary to breach the laterally-breachable portion of the guidewire-retaining segment can be at least 100 grams and no more than 20 kg. In various embodiments, the necessary force can be at least 500 grams and no more than 10 kg, or at least 1 kg and no more than 5 kg, or at least 1.5 kg and no more than 2.5 kg. Once the guidewire-retaining portion is completely breached, the force necessary to withdraw the tube from the anchored stent can be less than the force required to breach the guidewire-retaining portion.

We now refer to the figures, and in particular to FIG. 1, which shows a schematic illustration of a distal portion of a stent-conveyance tube 220 according to a non-limiting example. The tube 220 is elongated in that it is many times (e.g., 100 or 200 or more) times longer than it is thick. The tube 220 is shown as engaged with the distal end of a guidewire 12 for purposes of illustrating some of the features of the tube 220 and the manner in which it engages with the guidewire 12. Distal and proximal directions with respect to the tube and stent of FIG. 1 are shown by means of arrow 1001.

As shown in FIG. 1, a first distal segment 222 is demarcated by a distal aperture 320 and a proximal aperture 262. In some embodiments, the distal aperture is not necessarily at the distal tip (the tip of the distal portion of the tube 220) but rather is displaced proximally therefrom. In such embodiments, the distal aperture 320 preferably faces distally, or within 15° or 30° or 45° of the distal direction. Similarly, the proximal aperture 262 preferably faces proximally, as shown in FIG. 1, or within 15° or 30° or 45° of the proximal direction. A main purpose of the engagement of the tube 220 with the guidewire 12 is to enable distal advancement of the tube, along with one or more stents conveyed by the tube 220, to a target stent-deployment location within a lumen of a subject's body; therefore it can be desirable for the guidewire-engaged segment of the tube (with the guidewire engaged within) to traverse the length of the guidewire with a minimum of resistance, and a suitable angling of the apertures 320, 262 can contribute to the lowering of resistance from frictional and other forces. The guidewire-engaging segment 222 is also called a guidewire-retaining segment in this disclosure because the segment is designed to retain the guidewire therewithin as the tube traverses the guidewire. The guidewire-retaining segment 222 includes a lengthways laterally-breachable portion 278 which is configured to enable the retained guidewire 12 to breach the guidewire-retaining segment 222 laterally when a suitable shearing force is applied, as will be discussed in further detail hereinbelow.

It can be seen in FIG. 1 that a segment 232 proximal to the guidewire-retaining segment 222 has a diameter less than that of the guidewire-retaining segment, and it can be readily understood that the difference in diameters enables use of a straightforward design for the proximal aperture 262, i.e., formed as to face proximally as in the FIG. 1 example. In other examples of a stent-conveyance tube 222 according to embodiments (not illustrated), the diameter of proximal segment 232 can be the same as or greater than the diameter of guidewire-retaining segment 222, and the proximal aperture 262 can be positioned and angled accordingly. In any case, after exiting the guidewire-retaining segment 222 via the proximal aperture, the guidewire 12 extends proximally outside the tube 222 and does not interiorly traverse the next segment 232.

Referring now to FIGS. 2A and 2B, examples of configuring the distal end of a stent assembly 101 are illustrated schematically. The stent assembly 101 includes the stent-conveyance tube 220 of FIG. 1 together with a stent 52 mounted on the tube 220 so as to surround a segment of the tube 220. In some embodiments, the stent assembly additionally includes the guidewire 12. The stent 52 is displaced proximally from the guidewire-retaining segment 222, and is not necessarily contiguous to the guidewire-retaining segment 222, i.e., in the example shown there is an intervening segment 232 as was shown in FIG. 1. As shown in FIGS. 2A and 2B, the guidewire 12 extends proximally outside of the tube 220 after exiting the proximal aperture 262 and continues proximally alongside the stent 52.

The differences between the examples of FIG. 2A and FIG. 2B are to be found in the specific cross-section design of segment 232 (and of more proximal segments of the tube 220), and in the specific design of the laterally-breachable portion 278.

With respect to the cross-section of segment 232, in the example shown in FIG. 2A, the cross-section of the tube 220 at segment 232 (as shown in Detail Box 98) is open and U-shaped. In other words, proximal to the guidewire-retaining segment 222, the tube need not be a complete cylinder. The respective section of the tube can be formed with a open U-shape, or can be a collapsed or crushed segment of a completely cylindrical segment. Use of the U-shape can be helpful in some designs for facilitating the routing of guidewire 12 as it exits the proximal aperture 262. In contrast to the U-shape example of FIG. 2A, the cross-section of the tube 220 at segment 232 is a complete circle in the example shown in FIG. 2B, i.e., the tube 220 is shaped as a complete cylinder in segment 232. The segment of the tube 220 surrounded by the stent 52 can likewise employ either cross-section, and can simply continue the design choice of intermediating segment 232. Neither segment is limited to the specific designs illustrated in the non-limiting examples of FIGS. 2A and 2B, and any cross-sectional design can be employed.

With respect to the design of the laterally breachable portion 278 of the guidewire-retaining segment 232, the first of the two cross-sectional detail boxes labeled A1 in FIG. 2A shows that the laterally breachable portion 278 can include a slit 280 shaped to define two ‘lips’ 282 (‘slit lips’) that are in contact with each other, or nearly in contact with each other but not farther apart than the diameter of the guidewire 12, to define a closed-slit configuration in the absence of any forces applied to slit lips 282. The slit lips 282, are displaceable from each other, typically by suitable application of a force to cause the displacement, to define an opened-slit configuration. The second of the two cross-sectional detail boxes labeled A1 in FIG. 2A shows that the laterally breachable portion 278 can include a weakened sidewall portion 284, which is splittable/breachable by the guidewire 12 in response to a suitably applied force. A third example of a design of the laterally breachable portion 278 is shown in the detail box in FIG. 2B, in which the laterally breachable portion 278 is shown to be perforated, and thus splittable/breachable by the guidewire 12 in response to a suitably applied force. FIG. 2C shows the distal tip of a stent-conveyance tube 220 according to embodiments. The design of FIG. 2C includes a laterally breachable portion 278 embodied as a perforated portion of the guidewire-retaining segment 222 and thus is conceptually similar to the design illustrated in FIG. 2B.

Design of the laterally breachable portion 278 is not limited to the specific designs illustrated in the non-limiting examples of FIGS. 2A and 2B, and any functionally equivalent design that suitably enables the lateral breaching of the guidewire-retaining segment 222 can be used.

One or more anchor flaps 522 are formed on the external surface of the stent 52, as shown in FIGS. 2A-2B, so that when the stent 52 is deployed at a target location within a lumen of the subject, the one or more anchor flaps are effective to anchor the stent in place by catching or snagging on the interior wall of the lumen.

Referring now to FIG. 3, a method is disclosed for deploying a stent in a lumen of a human body. As illustrated by the flow chart in FIG. 3, the method comprises:

Step S01 passing an end, e.g., a proximal end, of the guidewire 12 through the guidewire-retaining segment 222 of the stent-conveyance tube 220 via respective distal and proximal apertures 320, 262; and

Step S02 arranging the stent 52 on the stent-conveyance tube 220, proximal to the guidewire-retaining segment 222. As mentioned earlier, there can be an additional segment 232 intermediating between the guidewire-retaining segment 222 and the tube segment on which stent 52 is mounted.

As shown in the flowchart, Steps S01 and S02 can be carried out in either order, i.e., first Step S01 and then Step S02, or first Step S02 and then Step S01. As an example, it may be desirable to have the tube 220 engaged with the guidewire 12 before mounting the stent 52 on the tube 220. As another example, it may be desirable to have the stent 52 in place on the tube 220 before engaging the tube 220 with the guidewire 12.

After carrying out Steps S01 and S02, the stent assembly 101 can be seen to be in a ‘stent-advancement configuration’ in which the guidewire 12 passes through the distal and proximal apertures 320, 262 so as to interiorly traverse the guidewire-retaining segment 222, and the stent 52 is arranged to surround a stent-conveyance tube segment that is proximally displaced from the guidewire-retaining segment 222, for advancement of the stent 52 together with the stent-conveyance tube 220 along the guidewire 12 into a body lumen of a human subject.

The method additionally comprises:

Step S03 advancing the stent-conveyance tube 220 along the guidewire 12 together with the stent 12, i.e., with guidewire-retaining segment 222 engaged with the guidewire 12, to deliver the stent 52 to a target location in a lumen, e.g., a bile duct, of a patient; and

Step S04 proximally withdrawing the stent-conveyance tube 220 to deploy the stent in the lumen, without manipulating the guidewire. The term ‘without manipulating the guidewire’ describes a situation wherein the stent 52 is anchored in the lumen by one or more anchor flaps 522 and is substantially immobilized (e.g., won't move longitudinally more than 1 mm, or more than 2 mm, or more than 3 mm, or more than 5 mm, or more than 10 mm) so as to resist longitudinal forces associated with withdrawing the stent-conveyance tube 220.

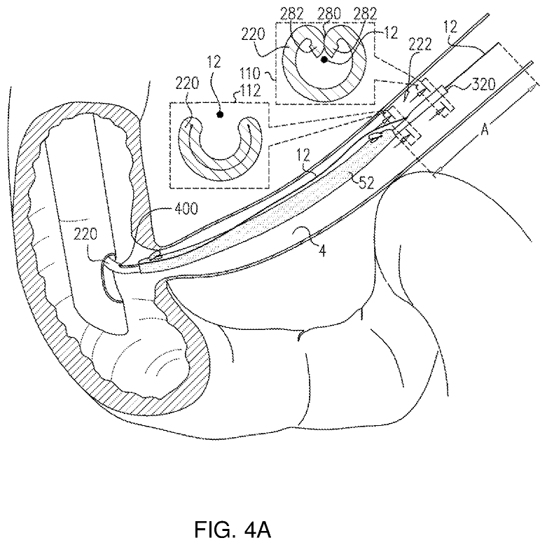

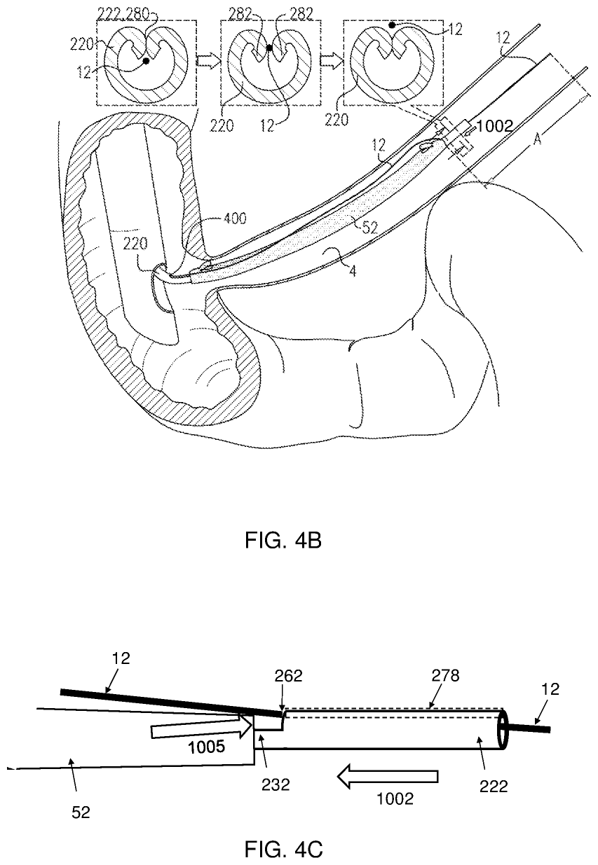

Steps of the instant method for deploying a stent in a lumen of a human body using a stent assembly 101, according to embodiments of the present invention, will be explained in greater detail in connection with FIGS. 4A-C.

FIG. 4A shows, schematically, a stent assembly 101 such as any one the stent assembly 101 of FIGS. 2A-2B upon ‘arrival’ of the stent 52 at a target location, with the stent assembly 101 still in the ‘stent-advancement configuration’ as described hereinabove, in a lumen 4 of a patient. In terms of the method steps described in the preceding paragraphs, FIGS. 2A-2B are ‘snapshots’ of a stent assembly 101 after carrying out Steps S01 and S02, and FIG. 4A is a snapshot after carrying out Step S03.

In the particular example of the stent assembly 101 illustrated in FIG. 4A the tube segment 232 proximal to the guidewire-retaining segment 222 has the ‘crushed U-shape’ cross-section shown in FIG. 2A, and uses the slit 280/‘slit-lips’ 282 design option for the laterally breachable portion 278 of the guidewire-retaining segment 222, as also shown in FIG. 2A. Selection of this design example throughout FIGS. 4A-4B is for convenience only, and any of the design options of FIGS. 2A-2B, or their functional equivalents, can be employed to equal benefit.

Unlike the illustrations of FIGS. 2A-2B, FIG. 4A shows the proximal end of the stent 52, and shows the guidewire 12 re-entering the interior of the tube stent-conveyance tube 220 through aperture 400. While this configuration is optional, it can be desirable to re-engage the guidewire 12 behind (i.e., proximal to) the stent 52 in the stent-advancement configuration. As a non-limiting example, this configuration can simplify control of the guidewire and stent-conveyance tube both during stent advancement and during later withdrawal of the tube and/or guidewire. The passage of the guidewire 12 also restricts the ability of the stent 52 to slip proximally with respect to the tube 220 during the stent advancement step S03. In addition, as will be discussed hereinbelow, using the configuration can be advantageous if it is desired to deliver a second stent together with (i.e., immediately or soon after, and alongside) delivery of the ‘first’ stent.

We now refer to FIGS. 4B and 4C, which schematically illustrate the dynamic of Step S04. Step S04 preferably is carried out after the stent assembly 101 has been advanced along the guidewire 12 until the stent 52 is at a target location, for example, one that may have been selected in advance, or one that may have been selected by using an endoscope inserted along guidewire 12 or another guidewire. In Step S04, the stent-conveyance tube 220 is withdrawn proximally, i.e., in the direction of arrow 1002 of FIGS. 4B and 4C. Specifically, the withdrawal of the stent-conveyance tube 220 is illustrated in FIG. 4B when the movement of the stent 52 is externally constrained. In the non-limiting example of FIG. 4B, the constraining is show as being due to the engagement of the stent 52 with a wall of the lumen 4. The stent 52, as noted hereinabove, is configured to be anchored in a wall of lumen 4 by anchor flap 522, or at least prevented from proximal travel of more than 1 mm, of more than 2 mm, of more than 3 mm, or more than 5 mm. As indicated in FIG. 4C, the travel of the stent-conveyance tube 220 in the proximal direction causes aperture 262, through which the guidewire 12 exits the guidewire-retaining segment 222, to approach the distal end of stent 52. This approaching causes a shearing force (schematically represented by arrow 1005) to impinge upon the portion of the guidewire 12 proximal to aperture 262 and eventually of the portion of the guidewire 12 distal to aperture 262. When the force applied in order to proximally withdraw the stent-conveyance tube 220 is in a suitable range, or alternatively (or equivalently) above a minimally necessary level of force, the shearing force starting at aperture 262 causes the guidewire 12 to breach the laterally-breachable portion 278 of the guidewire-retaining segment 222. Suitable ranges of force to be applied to proximally withdraw the tube 220 so as to breach the laterally-breachable portion 278 include: at least 100 grams and no more than 20 kg, or at least 500 grams and no more than 10 kg, or at least 1 kg and no more than 5 kg, or at least 1.5 kg and no more than 2.5 kg. Once the sidewall of the tube 220 in the guidewire-retaining segment 222, i.e., the laterally breachable portion, is breached, the guidewire 12 exits the guidewire-retaining segment 222 laterally and thus decouples or disengages from the tube 220. The breaching is shown in the three cross-sectional detail boxes of FIG. 4B, each representing a point in time, as follows: in the leftmost detail box, the guidewire 12 is still in the interior of the guidewire-retaining segment 222; in the center detail box, the guidewire 12 can be seen actively breaching the laterally-breachable portion; and in the rightmost detail box, the guidewire 12 can be seen to be outside of the guidewire-retaining segment 222 having exited laterally therefrom. Thus, the disengagement of the stent 52 from the stent-conveyance tube 220 takes place without the need for manipulation of the guidewire 12. In particular, there is no need for causing distal or proximal movement of the guidewire 12 in order to carry out the methods disclosed herein. Some inadvertent movement of the guidewire 12 may occur during or after Step 04, for example from incidental contact or friction with the withdrawing tube 220, although such movement is not substantive and does not impinge upon the scope of the invention which excludes necessitating any directed movement or manipulation of the guidewire 12.

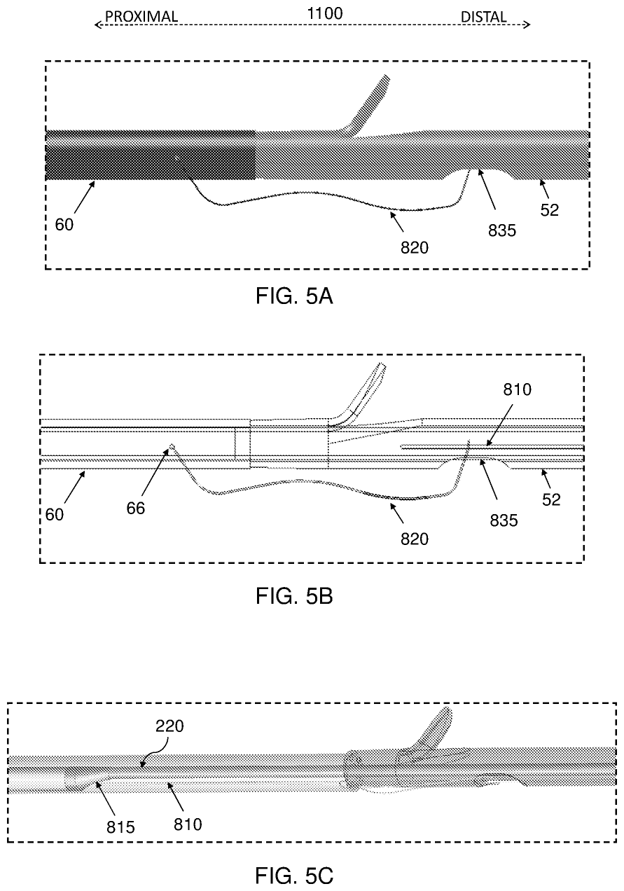

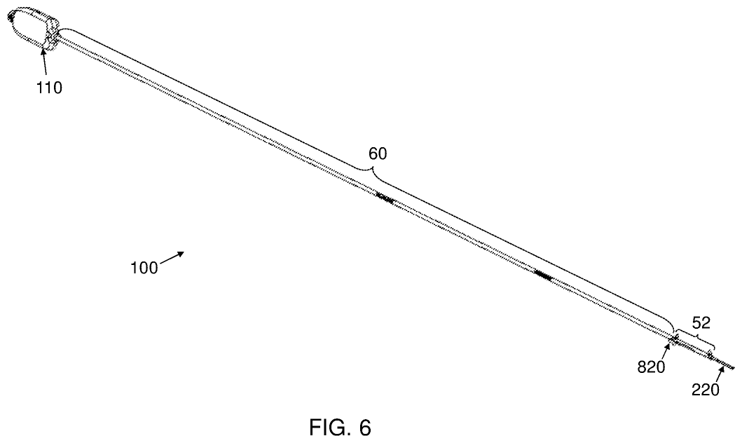

We now refer to FIGS. 5A, 5B, and 5C, which illustrate a proximally withdrawable locking mechanism for a stent-deployment assembly, e.g., the stent-deployment assembly 100 of FIG. 6. FIG. 5A shows an exterior view such that the locking mechanism is only partially visible. As can be seen, the proximal end of the stent 52 is arranged to slightly enter the pushing tube 60, and this is included within the scope of the pushing tube 60 being distally displaced from the stent 52. FIGS. 5B and 5C are, respectively, line and ‘transparent’ drawings illustrating additional features. FIGS. 5A and 5B show the same detail section of the stent-deployment assembly while FIG. 5C shows a longer detail section of a stent-deployment assembly.

The locking mechanism is effective to maintain a position of a stent 52 relative to the stent-conveyance tube 220 when the stent-deployment assembly is advanced along the guidewire 12. The locking mechanism is also effective to maintain a position of a stent 52 relative to the pushing tube 60.

A stent 52 is arranged to surround a first portion of a stent-conveyance tube 220. (shown in FIG. 5C). A pushing tube 60 is arranged to surround a second portion of a stent-conveyance tube 220 that is proximally displaced from the first portion, such that the pushing tube 60 is proximally displaced from the stent 52, although as described hereinabove the displacement can include incidental or de minimis overlap or even substantial overlap so long as any overlap does not interfere with the arrangement and function of the locking mechanism or with the disengagement of the stent 52 from the stent-conveyance tube 220. The distal and proximal directions for FIGS. 5A-5C are indicated by arrow 1100. A first locking member 810 is engaged at a proximal end with the stent-conveyance tube 220 at engagement point 815 (shown in FIG. 5C). An example of a suitable first locking member 810 is a wire comprising a metal or a metal alloy. The engagement of the first locking member 810 with the stent-conveyance tube 220 can include any kind of permanent or temporary attachment. In another example, the first locking member 810 can be permanently affixed to the stent-conveyance tube 220, e.g., by gluing or welding (all examples herein being illustrative and non-limiting). In another example, the first locking member 810 can be detachably attached to the stent-conveyance tube 220, e.g., by snapping or screwing two parts together, or by passing the first locking member 810 or an extension thereof through a hole or around the stent-conveyance tube 220. In some embodiments, the first locking member 810 can be formed as an integral part of the stent-conveyance tube 220 and does not require further attachment.

A second locking member 820 enters the pushing tube 60 through a hole 66, traverses a portion of the pushing tube 60 transversely, and exits through a second hole 66 (not visible in FIGS. 9A-9B). A loop portion of the second locking member 820 which is external to the pushing tube 60, is threaded under the first locking member 810 in stent-opening 835. The loop portion of the second locking member 820 is illustrated schematically as a loose loop but in actual implementation is preferably a tight loop so as to maintain the position of stent 52 when the stent-conveyance tube 220 is advanced along a guidewire 12 (not shown in FIGS. 5A-5C) together with the stent 52 and the pushing tube 60. The loop portion is preferably installed tightly enough that the presence of the second locking member 820 doesn't allow the stent-conveyance tube 220 and the pushing tube to separate from each other at all (or by more than 1 mm, or by more than 2 mm) as the loop portion is constrained by the first locking member 810 within an interior volume of the stent 52 accessible through the hole 835, e.g., ‘trapped’ between the first locking member 810 and a proximal edge of the hole 835. The design of the proximally withdrawable locking mechanism is such that a proximal withdrawal of the stent-conveyance tube 220 is effective to pull the first locking member 810 (which is engaged with the stent-conveyance tube 220) proximally, so as to disengage (free) the loop portion of the second locking member 820 so that the stent 52 and pushing tube 60 are freely separable. In other words, the locking mechanism is disengaged by the proximal withdrawal of the stent-conveyance tube 220. The stent 52 is thus deployable or advanceable without being engaged with the pushing tube 60.

The skilled artisan will understand that if the stent-deployment assembly 100 employs a stent-conveyance tube 220 having a guidewire-retaining segment 222 that includes a lengthways laterally-breachable portion 278 as illustrated, e.g., in FIGS. 1, 2C and 4C and discussed hereinabove in connection with those figures, then a proximal withdrawal of the stent-conveyance tube 200, e.g., with the stent 52 externally constrained, will be effective to both cause a guidewire 12 to breach the laterally-breachable portion 278 of the guidewire-retaining segment 222 so as to decouple the guidewire 12 from the tube 220 without manipulation of the guidewire 12, and (ii) disengage the proximally-withdrawable locking mechanism.

FIG. 6 shows an exemplary stent-deployment assembly 100 according to embodiments. A control element 110 can be provided for manipulation of the stent-conveyance tube 220 and/or for advancing the stent 52 using the pushing tube 60.

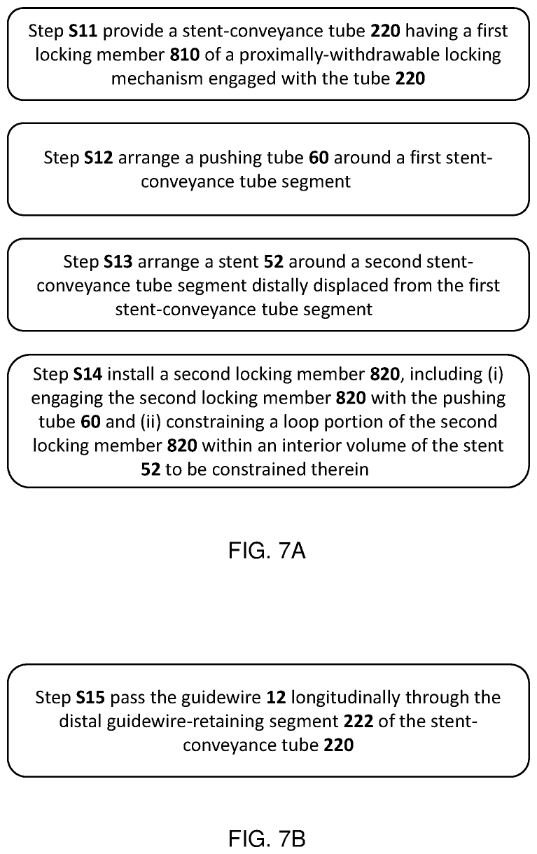

Referring now to FIG. 7A, a method is disclosed for assembling a stent-deployment assembly (or, equivalently, apparatus) 100. As illustrated by the flow chart in FIG. 7A, the method comprises the following steps:

Step S11: providing a stent-conveyance tube 220 having a first locking member 810 of a proximally-withdrawable locking mechanism engaged with the stent-conveyance tube 220. In some embodiments, the stent-conveyance tube 220 comprises a distal guidewire-retaining segment 222.

Step S12: arranging a pushing tube 60 around a first stent-conveyance tube segment.

Step S13: arranging a stent 52 around a second stent-conveyance tube segment that is proximally displaced from the guidewire-retaining segment 222, and distally displaced from the first stent-conveyance tube segment.

Step S14: installing a second locking member 820, wherein the installing includes (i) engaging the second locking member 820 with the pushing tube 60 and (ii) constraining a loop portion of the second locking member 820 within an interior volume of the stent 52 to be constrained therein.

In some embodiments, the method additionally comprises, as shown in the flowchart of FIG. 7B:

Step S15: passing a guidewire 12 longitudinally through the distal guidewire-retaining segment 222 of the stent-conveyance tube 220.

In some embodiments, Steps S11 through S14 are performed at a factory or assembly facility, while Step S15 is not performed at the factory or assembly facility.

According to embodiments, the method steps can be carried out in any order deemed practical for assembling the stent-deployment apparatus 100. In some embodiments, not all of the steps need be carried out.

It will be appreciated by persons skilled in the art that the present invention is not limited to what has been particularly shown and described hereinabove. Rather, the scope of the present invention includes both combinations and sub-combinations of the various features described hereinabove, as well as variations and modifications thereof that are not in the prior art, which would occur to persons skilled in the art upon reading the foregoing description.

1. A stent-deployment assembly for use with a guidewire, comprising:
a. an elongated stent-conveyance tube comprising a guidewire-retaining segment, the guidewire-retaining segment (i) including a lengthways laterally-breachable portion and (ii) being configured for having the guidewire traverse longitudinally therethrough;
b. a stent arranged to surround a stent-conveyance tube segment that is proximally displaced from the guidewire-retaining segment;
c. a pushing tube surrounding the stent-conveyance tube segment and proximally displaced from the stent; and
d. a proximally-withdrawable locking mechanism, proximally engaging the stent with the pushing tube so as to constrain distal movement of the stent relative to the pushing tube,
wherein, when movement of the stent is externally constrained, a proximal-direction withdrawal of the stent-conveyance tube is effective to (i) cause the guidewire to breach the laterally-breachable portion of the guidewire-retaining segment so as to decouple the guidewire from the tube without manipulation of the guidewire, and (ii) disengage the proximally-withdrawable locking mechanism.

2. The stent-deployment assembly of claim 1, wherein a proximal-direction withdrawal of the stent-conveyance tube effective to cause the guidewire to breach the laterally-breachable portion of the guidewire-retaining segment can be effected by applying a proximal-withdrawal force of at least 100 grams and no more than 20 kg.

3. The stent-deployment assembly of either one of claim 1 or 2, additionally comprising the guidewire.

4. The stent-deployment assembly of any preceding claim, wherein the proximally-withdrawable locking mechanism includes a first locking member proximally engaged with the elongated stent-conveyance tube.

5. The stent-deployment assembly of claim 4, wherein the first locking member is integrally formed with the elongated stent-conveyance tube.

6. The stent-deployment assembly of claim 4, wherein the first locking member is fixedly attached to the elongated stent-conveyance tube.

7. The stent-deployment assembly of claim 4, wherein the first locking member is detachably attached to the elongated stent-conveyance tube.

8. The stent-deployment assembly of any preceding claim, wherein the proximally-withdrawable locking mechanism includes a second locking member comprising a loop portion.

9. The stent-deployment assembly of claim 8, wherein the second locking member transversely traverses the pushing tube.

10. The stent-deployment assembly of either one of claim 8 or 9, wherein a portion of the second locking member is constrained within an interior volume of the stent by the presence of the first locking member.

11. The stent-deployment assembly of claim 10, wherein the disengaging of the proximally-withdrawable locking mechanism includes proximally withdrawing the first locking member so as to no longer constrain the portion of the second locking member.

12. A method of assembling a stent-deployment apparatus, the method comprising:
a. providing a stent-conveyance tube comprising a distal guidewire-retaining segment, the stent-conveyance tube having a first locking member of a proximally-withdrawable locking mechanism engaged with the stent-conveyance tube;
b. arranging a pushing tube around a first stent-conveyance tube segment;
c. arranging a stent around a second stent-conveyance tube segment that is proximally displaced from the guidewire-retaining segment and distally displaced from the first stent-conveyance tube segment; and
d. installing a second locking member, wherein the installing includes (i) engaging the second locking member with the pushing tube and (ii) constraining a loop portion of the second locking member within an interior volume of the stent to be constrained therein by the presence of the first locking mechanism.

13. The method of claim 12, additionally comprising: passing the guidewire longitudinally through the distal guidewire-retaining segment of the stent-conveyance tube.

14. The method of claim 13, wherein the passing through of the guidewire is not factory-performed and all other steps are factory-performed.

15. The method of any one of claims 12 to 14, wherein the first locking member is integrally formed with the stent-conveyance tube.

16. The method of any one of claims 12 to 14, wherein the first locking member is fixedly attached to the stent-conveyance tube.

17. The method of any one of claims 12 to 14, wherein the first locking member is detachably attached to the stent-conveyance tube.

18. A stent-deployment assembly comprising:
a. an elongated stent-conveyance tube comprising a hollow longitudinal lumen configured to have a guidewire traverse longitudinally therethrough;
b. a stent arranged to surround a first stent-conveyance tube segment;
c. a pushing tube surrounding a second stent-conveyance tube segment that is proximally displaced from the first stent-conveyance tube segment;
d. a first locking member engaged with the elongated stent-conveyance tube; and
e. a second locking member comprising a first portion arranged to transversely traverse the pushing tube and a second portion constrained within an interior volume of the stent by the presence of the first locking member.

19. The stent-deployment assembly of claim 18, wherein the first and second locking members are effective in combination to constrain distal movement of the stent relative to the pushing tube.

20. The stent-deployment assembly of either one of claim 18 or 19, wherein when movement of the stent is externally constrained, a proximal-direction withdrawal of the stent-conveyance tube is effective to cause the second locking member to no longer be constrained by the first locking member.

21. The stent-deployment assembly of any one of claims 18 to 20, wherein the first locking member is integrally formed with the elongated stent-conveyance tube.

22. The stent-deployment assembly of any one of claims 18 to 20, wherein the first locking member is fixedly attached to the elongated stent-conveyance tube.

23. The stent-deployment assembly of any one of claims 18 to 20, wherein the first locking member is detachably attached to the elongated stent-conveyance tube.
